(19) United States
(12) Patent Application Publication
(10) Pub. No.: US 2014/0299484 A1
(43) Pub. Date: Oct. 9, 2014

(54) Medical Kit With Visually Enhanced Outer Layer For Accommodating Equipment With Visually Enhanced Removable Protective Coverings

(71) Applicant: Cambridge Sensors USA LLC, Plainfield, IL (US)

(72) Inventor: Frederick W. Engimann, III, Plainfield, IL (US)

(21) Appl. No.: 14/245,980

(22) Filed: Apr. 4, 2014

Related U.S. Application Data
(60) Provisional application No. 61/808,617, filed on Apr. 4, 2013.

(51) Int. Cl. A61B 19/02; A61B 5/15; A61B 5/145

(52) U.S. Cl. CPC A61B 19/026; A61B 5/14532; A61B 5/1411
USPC 205/782; 205/792; 206/305

(57) ABSTRACT

A resilient sleeve removably encases a body fluid monitor. The sleeve and a corresponding medical kit include a visually enhancing adornment for promoting the optical recognizability of the encased body fluid monitor and the kit. This is useful in medical emergencies when locating the body fluid monitor can be the difference between life and death. The visually enhancing adornment can include a highly visible color and/or indicia such as a letter, word, phrase and/or logo. In some embodiments the highly visible color is contrastable to blood and/or orange. In some embodiments the color of the inside of the kit is in contrast to the color of the resilient sleeve.

### CROSS-REFERENCE TO RELATED APPLICATION

This application relates to and claims priority benefits from U.S. Provisional Application No. 61/808,617 having a filing date of Apr. 4, 2013. The '617 provisional application is incorporated herein by reference in its entirety.

### Field of the Invention

The present invention relates to medical devices and, in particular, to resilient sleeves for removably encasing medical equipment and a corresponding kit that contain a visually enhancing adornment to increase their optical recognizability.

### BACKGROUND OF THE INVENTION

In emergency medical situations, assessing a patient's condition quickly and accurately can make the difference between successful recovery and tragedy. When responding to an emergency call, emergency medical technicians (EMTs) must be able to quickly identify the instruments and equipment appropriate for the situation, and then deploy them to properly diagnose and stabilize the patient. Identifying the appropriate instruments and equipment can be challenging because of the amount of equipment carried in emergency vehicles and portable cases that EMTs use to carry that equipment.

In many cases, body fluid monitors must be utilized to assess a patient's conditions. Examples of body fluid monitors include, but are not limited to, devices that test an individual's blood for total cholesterol, HDL, triglyceride, ketone, glucose, and lipid panel levels, metabolic syndrome, and the individual's prothrombin ratio (PR) and international normalized ratio (INR). Other examples of body fluid monitors include portable AIDS and other disease detectors, many pregnancy tests and urine analyzers.

In particular, one of the body fluid monitors typically deployed in medical emergencies is a blood constituent meter, most commonly a blood glucose meter. Blood glucose meters are typically hand-held devices that accept insertable test strips into which a drop of blood from a patient is introduced. The insertable test strip promotes an electrochemical reaction with the blood that generates an electronic signal, which the blood glucose meter processes to calculate and display the concentration of glucose in the patient's blood. Knowing whether or not the blood glucose concentration is within a normal range can assist in the diagnosis of a patient's condition.

As the technology underlying body fluid monitors has advanced, the monitors have become more efficient, compact, and precise. Although more convenient to handle, the smaller sized monitors are more difficult to locate among the many other instruments and equipment carried to an emergency call. Up to now, body fluid monitors and their carrying cases, especially blood glucose meters, have been made in dark colors because of the occasional droplet of blood that can accidentally drip or be wiped on the surface of the meter and/or its carrying case. The dark color of the meter and case masks the presence of such residual blood which, if readily visible, could be disturbing to patients and dirt stains which could signal that the monitor and case have not been properly cleaned or maintained. As a result, body fluid monitors and their carrying cases have not been made in highly visible and/or contrasting colors, but have instead been designed to render the monitors and carrying cases indistinguishable in color or appearance from bodily fluids, particularly blood and dirt.

The absence of visually enhancing color systems for body fluid monitors, while advantageous for masking the presence of residual blood and dirt, is disadvantageous in medical emergencies or otherwise urgent medical situations. In such time-sensitive situations, a visually enhancing color can make the body fluid monitor and/or its carrying case more readily recognizable and permit the EMT to save time in deploying the monitor to assist in diagnosing the patient. In these situations, the desire for a dark colored surface to mask the presence of residual blood or dirt is outweighed by the enhanced visibility and recognizability afforded by an optically distinguished surface.

### SUMMARY OF THE INVENTION

A resilient sleeve removably encases a body fluid monitor. The sleeve comprises a visually enhancing adornment for promoting the optical recognizability of the encased body fluid monitor.

In one embodiment of the body fluid monitor, the visually enhancing adornment comprises a color contrastable from the body fluid being tested. The visually enhancing adornment can also comprise indicia contrastable from the body fluid being tested. The indicia can comprise a visually recognizable pattern, which in turn can comprise at least one of a letter, a word, a phrase and a logo.

In one method of utilizing the visually resilient sleeve, a user has to measure one constituent in a body fluid in an emergency situation having a plurality of medical devices present for deployment to diagnose and stabilize a patient. The user, after assessing the patient's condition as warranting measurement of a body fluid constituent which is accomplished via the body fluid monitor encased in the removable resilient sleeve with the visually enhancing adornment, visually scans the plurality of medical devices in his vicinity. The user then identifies the body fluid monitor from among the plurality of medical device by recognizing the visually enhancing adornment and deploys the body fluid monitor to measure the body fluid constituent.

In one embodiment of the foregoing method, the visually enhancing adornment comprises a color contrastable from blood and/or dirt. The visually enhancing adornment can also comprise contrastable indicia. The indicia can be a visually recognizable pattern, which in turn can be a letter, a word, a phrase and/or a logo.

In one example the color of the sleeve and/or the indicia is a color contrastable from blood.

In some embodiments, the body fluid monitor is configured to be used by a veterinarian with animals, such as dogs, cats, horses.

In at least some embodiments of the foregoing method, a medical kit which is configured to hold the body fluid monitor is visually enhancing, either with respect to its color and/or an indicia such as a visually recognizable pattern, such as a letter, word, phrase and/or logo.

### BRIEF DESCRIPTION OF THE DRAWINGS

FIG. 1 is a perspective view of a resilient sleeve for removably encasing of a body fluid monitor with an insignia.

FIG. 2 is a perspective view of the resilient sleeve for removably encasing a body fluid monitor illustrated in FIG. 1 with the body fluid monitor inserted.

FIG. 3 is a top view of a body fluid monitoring kit, which includes a body fluid monitor, test strips, calibration fluid, alcohol wipes, and a lancing device and disposable lancets for drawing a drop of blood from a patient.

FIG. 4 is a perspective view of the front of the body fluid monitoring kit of FIG. 3.

FIG. 5 is a perspective view of the back of the body fluid monitoring kit of FIG. 3.

### DETAILED DESCRIPTION OF PREFERRED EMBODIMENT(S)

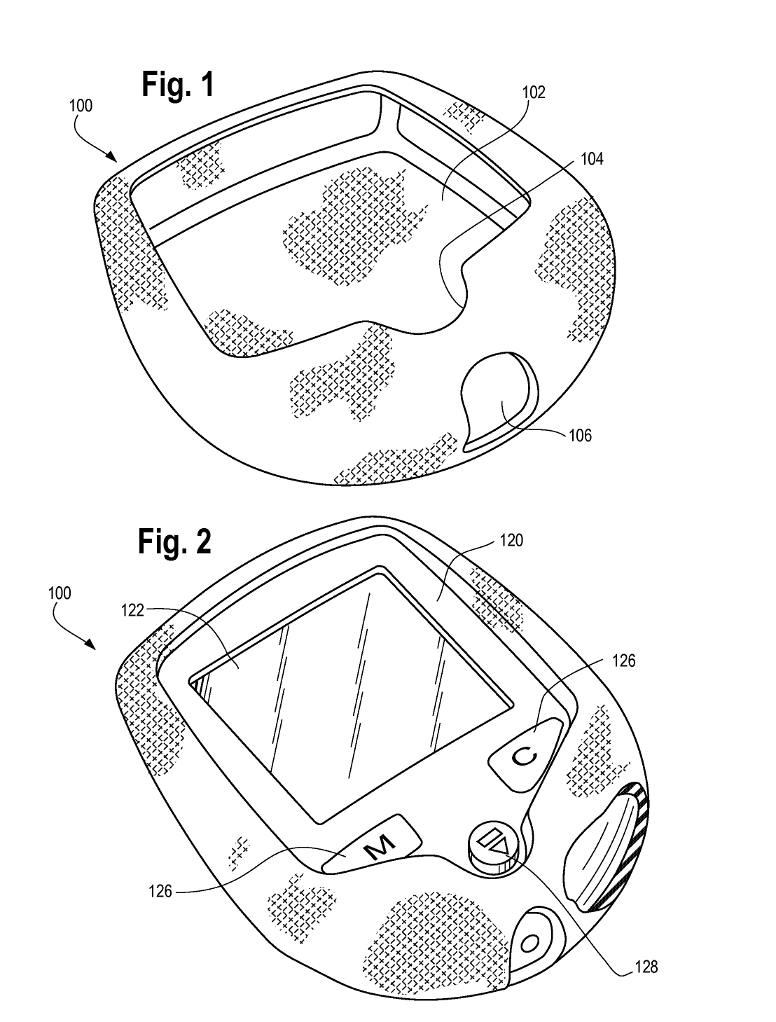

Turning first to FIGS. 1, sleeve 100 is capable of encasing body fluid monitor 120 illustrated in FIG. 2. Sleeve 100 is formed from a resilient material that is stretchable to wrap sleeve 100 around the edges of body fluid monitor 120. Suitable materials for sleeve 100 include natural and synthetic rubbers and/or elastomeric materials with thermal and physical stability, for example latex, styrene-butadiene rubbers, synthetic rubbers prepared from isoprene, chloroprene and isobutylene, either pure and/or mixed in various proportions to be copolymerized to produce resilient materials with physical, mechanical, and chemical properties suitable for the present resilient sleeve. The material should be easy to clean and disinfect, and should be compatible with the cleaning solutions and/or disinfecting chemicals. In some embodiments, the material of the sleeve is of such as nature that it protects body fluid monitor 120 from damage caused by drops, scratches, and/or spills.

In at least some embodiments, sleeve 100 is configured to be easily removable so it can be cleaned and/or disinfected between uses. In other embodiments, sleeve 100 is configured to be tightly sealed so as to waterproof the back of body fluid monitor 120.

Sleeve 100 can be made of a highly visual color to improve the ability of individuals to recognize it. In some embodiments, that color is contrastable to blood and/or dirt. Sleeve 100 can also contain indicia 135, which can also be a highly visual color. In some embodiments the color is contrastable to blood and/or dirt.

As shown in FIG. 1, sleeve 100 has a major opening 102, which when sleeve 100 encases body fluid monitor 120, as shown in FIG. 2, leaves body fluid monitor display 122 exposed and readable by the user. Major opening 102 in sleeve 100 also leaves buttons 124 and 126 on body fluid monitor 120 exposed. As further shown in FIGS. 1 and 2, sleeve 100 has a notch 104 formed therein in a location corresponding to one of the buttons on the face of the body fluid monitor that sleeve 100 is designed to encase. When body fluid monitor 120 in FIG. 2 is encased within sleeve 100, notch 104 leaves body fluid monitor button 128 uncovered and readily accessible for the user to press while performing the measurement process.

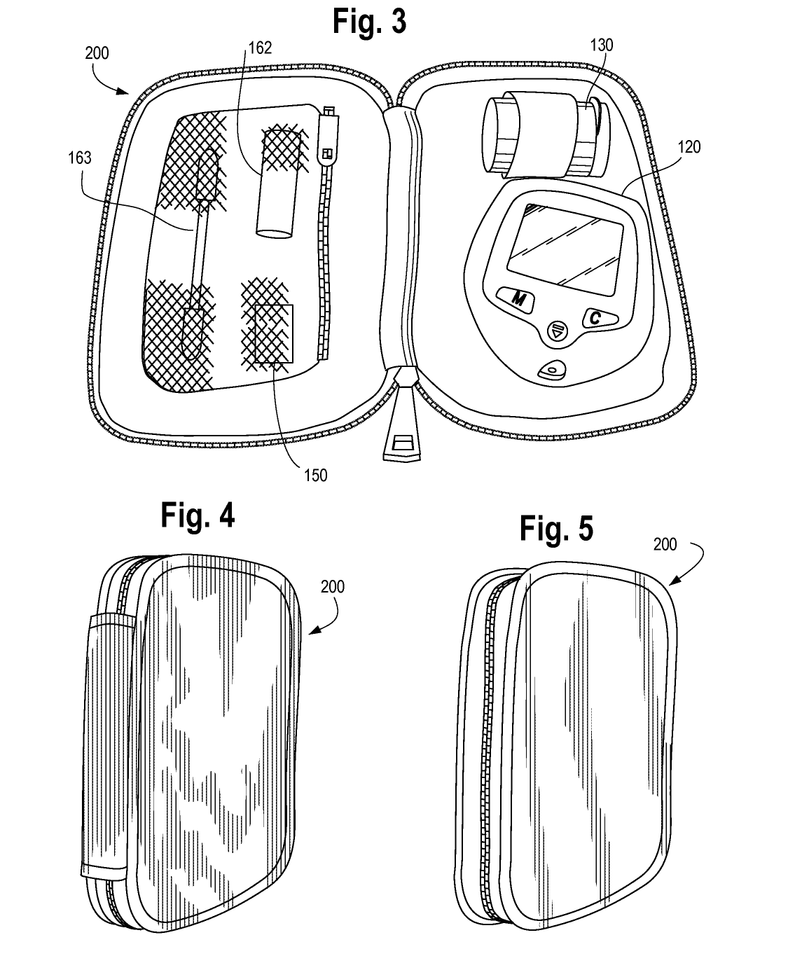

As specifically shown in FIGS. 1 and 2, sleeve 100 has a test strip opening 106 formed therein through which a test strip, such as test strip 130 shown in FIG. 3, can be inserted into body fluid monitor 120 of FIG. 2.

FIGS. 3, 4, and 5 illustrates body fluid monitoring kit 200, which includes a monitor 120 and associated components, including test strips 130, calibration fluid 150, lancing device 160 and disposable lancets 162 for drawing a drop of blood from a patient's finger. Lancets 162 and lancing device 160 can also be the color of sleeve 100 to make them more easily identifiable. In other embodiments, lancets 162 and/or lancing device 160 can be another contrasting color. In some embodiments, lancet 162 and/or lancing device 160 can contain an indicia. The indicia can be, among other things, a pattern, symbol, letter, word, number, and/or logo.

Body fluid monitoring kit 200 can match the color of sleeve 100 to make it more easily identifiable. In other embodiments, body fluid monitoring kit 200 can be another contrasting color. In some embodiments body fluid monitoring kit 200 contain an indicia. The indicia can be, among other things, a pattern, symbol, letter, word, number, and/or logo.

While particular elements, embodiments and applications of the present invention have been shown and described, it will be understood, that the invention is not limited thereto since modifications can be made by those skilled in the art without departing from the scope of the present disclosure, particularly in light of the foregoing teachings.

What is claimed is:

1. A system for increasing the optical recognizability of a medical kit containing a body fluid monitor comprising a removable encasing for said medical device, said encasing having first visually enhancing adornment for promoting optical recognizability of said medical device.

2. The system of claim 1, wherein said medical kit has second visually enhancing adornment for promoting the optical recognizability of said medical kit.

3. The system of claim 2 wherein each of said first and second adornments are a visible color.

4. The system of claim 3, wherein said visible color is contrastable with respect to the body fluid being monitored.

5. The system of claim 4, wherein the body fluid is blood.

6. The system of claim 4, wherein at least one of said visible colors is orange.

7. The system of claim 4, wherein at least one of said visible colors is pink.

8. The system of claim 2, wherein at least one of said first and said second adornments is perceivable indicia.

9. The system of claim 8, wherein said indicia is a pattern.

10. The system of claim 8, wherein said indicia is at least one of an alphabetic character and a numerical character.

11. The system of claim 10, wherein said indicia is a word.

12. The system of claim 8, wherein said indicia is a symbol.

13. The system of claim 1, wherein said body fluid monitor is a blood glucose meter.

14. The system of claim 13, wherein said medical kit further contains at least one lancet for piercing derma to draw a quantity of blood.

15. The system of claim 14, wherein said lancet bears said visible color of one of said first and second adornments.

16. A method for measuring a body fluid constituent comprising steps of:
(a) assessing a condition of a patient as warranting a measurement of a body fluid constituent wherein said measurement is effected via a body fluid monitor;
(b) visually scanning said an area for a medical kit containing said body fluid monitor; and
(c) identifying said medical kit by recognizing a visually enhancing adornment of said medical kit.

17. The method of claim 14, further comprising:
(d) deploying said medical device to measure said body fluid constituent.

18. The method of claim 14, wherein said adornment is a visible color.

19. The method of claim 16, wherein said visible color is contrastable with respect to the body fluid being monitored.

20. The method of claim 17, wherein the body fluid is blood.

21. The method of claim 17, wherein said visible color is orange.

22. The method of claim 17, wherein said visible color is pink.

23. The method of claim 14, wherein said adornment is perceivable indicia.

24. The method of claim 21, wherein said indicia is a pattern.

25. The method of claim 21, wherein said indicia is at least one of an alphabetic character and a numerical character.

26. The method of claim 23, wherein said indicia is a word.

27. The method of claim 21, wherein said indicia is a symbol.

28. The method of claim 14, wherein said body fluid monitor is a blood glucose meter.

29. The method of claim 28, wherein said medical kit further contains at least one lancet for piercing derma to draw a quantity of blood.

30. The method of claim 29, wherein said lancet bears said visible color of one of said first and second adornments.
